(19) United States
(12) Patent Application Publication
(10) Pub. No.: US 2024/0424235 A1
(43) Pub. Date: Dec. 26, 2024

(54) ULTRASONIC SENSING FOR RESPIRATORY MONITORING

(71) Applicant: TEXAS INSTRUMENTS INCORPORATED, Dallas, TX (US)

(72) Inventor: John VARELA MUNOZ, Plano, TX (US)

(21) Appl. No.: 18/340,621

(22) Filed: Jun. 23, 2023

(51) Int. Cl. A61M 16/00

(52) U.S. Cl. CPC A61M 16/024; A61M 16/0003; A61M 16/0069; A61M 2016/0039

(57) ABSTRACT

In an example, a system includes a flow element configured to direct a gas flow. The system also includes a temperature sensor coupled to the flow element, the temperature sensor configured to determine a temperature of the gas flow. The system includes a first ultrasonic transducer and a second ultrasonic transducer coupled to the flow element, where the first ultrasonic transducer and the second ultrasonic transducer are configured to determine a time of flight of the gas flow. The system also includes a processor configured to determine a volume concentration of a gas in the gas flow based at least in part of the time of flight and the temperature of the gas flow.

### BACKGROUND

Respiratory therapy devices, such as respirators or ventilators, have multiple sensors for monitoring a patient. The sensors may include flow sensors, concentration sensors, differential pressure sensors, temperature and humidity sensors, etc. Different technologies may be used for the various sensors, such as ultrasonic, thermal, mechanical, electrochemical, or optical.

### SUMMARY

In at least one example of the description, a method includes determining time of flight of a gas flow with a first ultrasonic transducer and a second ultrasonic transducer, where the gas flow includes a volume concentration of oxygen. The method also includes, responsive to determining the time of flight, determining a speed of sound in the gas flow. The method includes responsive to determining the speed of sound, determining a volume concentration of carbon dioxide in the gas flow based at least in part on the speed of sound and the volume concentration of oxygen.

In at least one example of the description, a system includes a flow element configured to direct a gas flow. The system also includes a temperature sensor coupled to the flow element, the temperature sensor configured to determine a temperature of the gas flow. The system includes a first ultrasonic transducer and a second ultrasonic transducer coupled to the flow element, where the first ultrasonic transducer and the second ultrasonic transducer are configured to determine a time of flight of the gas flow. The system also includes a processor configured to determine a volume concentration of a gas in the gas flow based at least in part of the time of flight and the temperature of the gas flow.

In at least one example of the description, a system includes a respiratory device. The system includes a flow element coupled to the respiratory device and configured to direct a gas flow, where the gas flow includes a volume concentration of oxygen. The system also includes a temperature sensor coupled to the flow element, the temperature sensor configured to determine a temperature of the gas flow. The system includes a first ultrasonic transducer and a second ultrasonic transducer coupled to the flow element, where the first ultrasonic transducer and the second ultrasonic transducer are configured to determine time of flight of the gas flow. The system also includes a processor configured to determine a speed of sound in the gas flow. Responsive to determining the speed of sound, the processor is further configured to determine a volume concentration of carbon dioxide in the gas flow based at least in part on the speed of sound and the volume concentration of oxygen.

### BRIEF DESCRIPTION OF THE DRAWINGS

FIG. 1A illustrates an example system for respiratory monitoring.

FIG. 1B illustrates an example flow element.

FIG. 2A is a graph illustrating an example time of flight.

FIG. 2B is a graph illustrating an example volume flow rate.

FIG. 3 is a graph illustrating an example speed of sound during inhalation and exhalation.

FIG. 4 is a graph illustrating an example concentration percentage versus speed of sound.

FIG. 5 is a graph illustrating an example slope versus inhalation speed of sound.

FIG. 6 is a flow diagram of an example method for determining volume concentration.

### DETAILED DESCRIPTION

Current respiratory therapy devices have multiple sensors for monitoring a patient. These respiratory devices may include respirators, ventilators, continuous positive airway pressure (CPAP) machines, etc. The sensors include flow sensors, concentration sensors, differential pressure sensors, and temperature and humidity sensors. Each additional sensor increases the size and cost of the sensing system. Some sensors may be inefficient, have limited lifetime, or have slow response time.

In examples herein, ultrasonic transducers are used for flow sensing in the respiratory device. The ultrasonic transducers perform time of flight measurements for flow sensing. With a combination of absolute and differential times of flight, temperature sensing, and humidity sensing, the examples herein may also determine oxygen (O₂) and carbon dioxide (CO₂) volume concentration (e.g., concentration) of a gas. Flow rate and differential pressure may also be determined using the ultrasonic transducers. Therefore, the ultrasonic transducers, combined with a temperature and humidity sensor, can provide four different sense measurements: flow rate, differential pressure, O₂ concentration, and CO₂ concentration. Separate sensors for each of these measurements may be replaced by the ultrasonic sensing system described herein. The systems and methods described herein may reduce size, reduce cost, increase efficiency, improve response time, and increase lifetime of a respiratory sensing system compared to previous solutions.

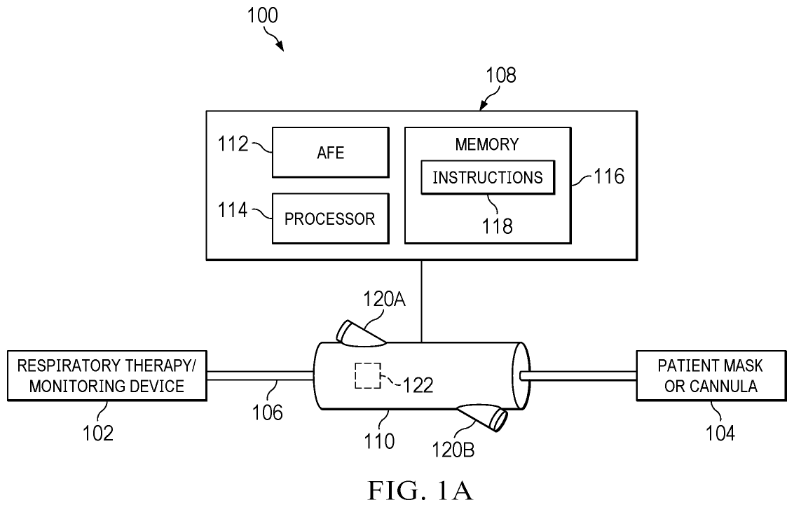

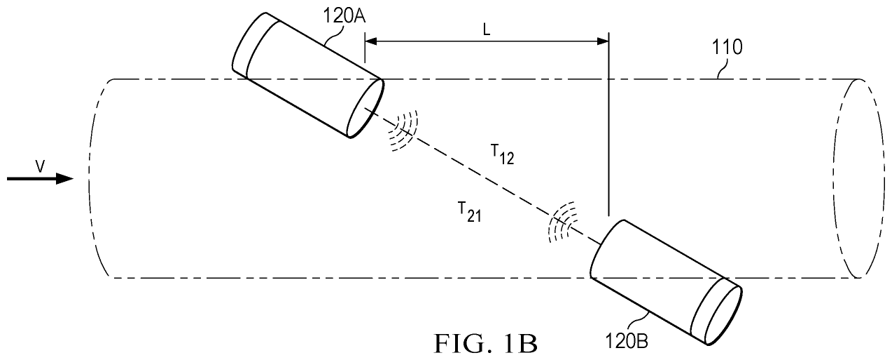

FIG. 1A illustrates an example system 100 for respiratory monitoring. System 100 includes a respiratory therapy or monitoring device 102, referred to herein as respiratory device 102. Respiratory device 102 may be a respirator, ventilator, CPAP machine, or the like. System 100 includes patient mask or cannula 104, tube 106, microcontroller unit (MCU) 108, and flow element 110. MCU 108 includes an analog front end (AFE) 112, a processor 114, memory 116, and instructions 118. Flow element 110 includes a first transducer 120A and a second transducer 120B (referred to collectively as transducers 120, or individually as a transducer 120). Flow element 110 also includes a temperature and humidity sensor 122, referred to herein as temperature sensor 122.

Respiratory device 102 is coupled to a patient mask or cannula 104 via a tube 106. In one example, cannula 104 is a nasal cannula that provides O₂ and/or medication to a patient. The flow element 110 may be coupled to tube 106 and MCU 108.

MCU 108 is any MCU that includes the hardware and/or software to perform the operations described herein. MCU 108 may be a digital signal processor (DSP), a DSP+MCU processor, a field programmable gate array (FPGA), or an application specific integrated circuit (ASIC). MCU 108 includes an analog front end (AFE) 112, a processor 114, memory 116, and instructions 118. AFE 112 includes any suitable circuitry for interfacing the sensors described herein to MCU 108. Processor 114 may perform all or portions of the methods described herein. Processor 114 as described herein may include any suitable processor or combination of processors. Processor 114 may be a central processing unit (CPU), DSP. ASIC, or other suitable hardware.

Memory 116 may include a single memory or multiple memory modules. Memory 116 provides storage, e.g., a non-transitory computer readable medium, which may be used to store software instructions 118 executed by a processor 114, such as any software instructions for implementing the operations described herein. A memory 116 may include any suitable combination of read-only memory (ROM) and/or random access memory (RAM), e.g., static RAM.

Memory 116 may include any suitable data, code, logic, or instructions 118. Processor 114 reads and executes computer-readable instructions 118. The processor 114 may invoke and execute instructions in a program stored in the memory 116, including instructions 118. Instructions 118 may perform the actions described herein, such as determining flow rate, differential pressure, O₂ concentration, and CO₂ concentration.

Memory 116 may be integrated with the processor 114. Memory 116 stores the instructions 118 for implementing the various methods and processes provided in the various examples of this description. A processor 114 described herein may use any combination of dedicated hardware and instructions stored in a non-transitory medium, such as memory 116. The non-transitory medium includes all electronic mediums or media of storage, except signals. Examples of suitable non-transitory computer-readable media include one or more flash memory devices, battery-backed RAM, solid state drives (SSDs), hard disk drives (HDDs), optical media, and/or other memory devices suitable for storing the instructions 118 for the processor 114.

System 100 also includes flow element 110. Flow element 110 receives and directs the gas flow in tube 106 between respiratory device 102 and cannula 104. Flow element 110 includes the first transducer 120A, second transducer 120B, and temperature sensor 122.

Ultrasonic transducers 120 measure time of flight (TOF) by sending an ultrasonic pulse and then listening for echoes returning from the target object. Two measurements may be taken (one from each transducer 120) to determine a differential TOF, the process of which is described below. The two measurements are used to determine a velocity of the gas flow, and also used to determine a concentration of O₂. Also, pressure of the gas flow may be determined as described below. The concentration of CO₂ may also be determined using the transducers 120 and the temperature sensor 122 as described herein. Therefore, flow rate, differential pressure, O₂ concentration, and CO₂ concentration are determined herein without the use of separate sensors for each measurement.

In other examples, TOF is determined using any suitable sensors and/or techniques. Some techniques include sampling with an analog to digital converter, digital filtering, and various other signal processing techniques. Other examples include transmitting a pulse and receiving a return waveform, where amplitude detection and zero crossing are performed.

FIG. 1B illustrates an example flow element 110. Flow element 110 includes transducers 120A and 120B. Temperature sensor 122 is not shown in FIG. 1B. The arrow labeled “V” represents a velocity of the gas flow passing left to right through the flow element 110. “L” represents the x-vector of the sensor distance (e.g., the horizontal distance between transducers 120A and 120B).

In an example, two TOF measurements are taken to find a differential. Measuring a differential provides a result that is agnostic of the medium (e.g., the gas). For example, different gases have different molecular weights. Different molecular weights can affect the TOF, as well as the temperature. By measuring a differential, these differences are offset and do not have to be accounted for.

In one example operation, a TOF from transducer 120A to 120B is taken in a first instance. This TOF is referred to as T₁₂ (the time for the signal to travel from transducer 1 (120A) to transducer 2 (120B)). T₁₂ represents the speed of sound in the medium plus the velocity V of the gas flow. In a second instance, a TOF from transducer 120B to 120A is taken. This TOF is referred to as T₂₁ (the time for the signal to travel from transducer 2 (120B) to transducer 1 (120A)). T₂₁ represents the speed of sound in the medium minus the velocity V of the gas flow. These two measurements may be used to determine the velocity V of the gas flow, and also C (the speed of sound in the medium). The velocity V measurement is independent of temperature and pressure in most operating conditions.

In an example, T₁₂ is given by Equation (1):

$${T}_{12} = \frac{L}{C + V} \qquad ( 1 )$$

T₂₁ is given by Equation (2):

$${T}_{21} = \frac{L}{C - V} \qquad ( 2 )$$

Equations (1) and (2) may be solved for V with Equation (3):

$$V = \frac{L}{2} ( \frac{1}{{T}_{12}} - \frac{1}{{T}_{21}} ) = \frac{L}{2} ( \frac{Δ T}{{T}_{12} {T}_{21}} ) \qquad ( 3 )$$

where ΔT is the difference between T₁₂ and T₂₁. Equation (3) shows that the velocity V does not depend on C. After V is determined, Equations (1) or (2) may be used to solve for C.

The speed of sound C in the flow element is related to the molecular weight M of the gas mixture and temperature T. This relationship is based on the ideal gas law, and is fairly accurate in most ambient conditions. After C is determined as described above, the molar weight p of O₂ in an air mixture may be determined, where the air mixture is mostly a mix of nitrogen (N₂) and O₂. To simplify the calculation, the air mixture may be treated as a mix of N₂ and O₂, where the molecular weight of N₂ may be adjusted to account for the weight of the minor gases found in the air mixture.

To solve for the molar weight p of O₂ in an air mixture, C is determined from the times of flight as described above, and a temperature sensor determines a temperature T. Equation (4) is the equation for velocity of gas in a binary mixture (such as N₂ and O₂):

$$C = \sqrt{\frac{k R T}{M}} \qquad ( 4 )$$

M is the molar weight of the gas mixture, and k is the specific heat ratio, which is approximately 1.4 for O₂ and air. R is the universal gas constant, which is approximately 8.314 Joules K⁻¹ mol⁻¹ (where K is temperature in Kelvin and mol is moles). To solve for p, the molar weight of O₂, a substitution for M is performed to produce Equation (5):

$$C = \sqrt{\frac{k R T}{{M}_{N} ( 1 - ρ ) + {M}_{{O}_{2}} ρ}} \qquad ( 5 )$$

Where M_{N}(1−φ represents the fraction of N₂ (plus the trace amounts of other gases) in the air mixture, and Mₒ_{₂}ρ is the fraction of O₂ in the air mixture. In Equation (5), this expression for the speed of sound C uses a single value for k, the specific heat ratio. This expression is a simplified version that works for O₂ concentration in an air mixture, because both air (mainly O₂ and N₂) and O₂ have a k of ˜1.4. A non-simplified version of this expression is described below. Solving Equation (5) for p produces Equation (6):

$$ρ = \frac{\frac{k R T}{{C}^{2}} - {M}_{N}}{{M}_{{O}_{2}} - {M}_{N}} \qquad ( 6 )$$

Therefore, in an air mixture of mostly N₂ and O₂, the concentration p of O₂ may be determined with the ultrasonic transducers 120 and a temperature sensor 122 as described herein. This example eliminates the need for a separate O₂ sensor in the system. In other examples, humidity compensation could be performed for more precise measurements. However, a change in humidity from 0% to 100% produces an error of about 5% or less in one example, so the error without humidity compensation is generally small, and can be ignored in some examples.

The non-simplified version of Equation (5) is useful for gas mixtures that are not mostly air and O₂, such as with a CO₂ mixture. In a mixture such as a CO₂ mixture, the value of k is expanded into k₁ and k₂, where k₁ is the specific heat ratio of the air mixture, and k₂ is the specific heat ratio of the other gas or gases in the mixtures, such as CO₂. The non-simplified version of Equation 5 is shown in Equation (7):

$$C = \sqrt{\frac{R T [ {k}_{1} ( 1 - ρ ) + {k}_{2} ( ρ ) ]}{{M}_{1} ( 1 - ρ ) + {M}_{2} ( ρ )}} \qquad ( 7 )$$

Equation (7) may be solved for p, the molar weight of CO₂ in the gas mixture in this example. Solving Equation (7) for p yields Equation (8):

$$ρ = \frac{{k}_{1} - ( \frac{{C}^{2}}{R T} ) {M}_{1}}{( \frac{{C}^{2}}{R T} ) ( {M}_{2} - {M}_{1} ) + {K}_{1} - {K}_{2}} \qquad ( 8 )$$

Pressure may also be determined using the ultrasonic transducers 120. Pressure P generally does not affect the speed of sound C in a medium. Pressure and density are inversely proportional, so the changes to one tends to cancel out changes to the other with respect to C. Pressure P may be determined if the dimensions of flow element 110 are known along with the volumetric flow rate. The volumetric flow rate is determined from the velocity of the gas flow and the dimensions of the flow element 110. A differential pressure Δp is found with Equation (9):

$$Δ p = \frac{8 μ LQ}{π {R}^{4}} \qquad ( 9 )$$

where p is the pressure in Pascals (Pa) and u is the coefficient of viscosity in Pa*s, which is approximately 2.01 for a 90% O₂ mixture. L is the length of the pipe in meters (m), Q is the volumetric flow rate in cubic meters (m³) per second, and R is the radius of the pipe in m. The length L and radius R may be found using a volume equivalent model to account for flow element 110 and tube 106 for a particular system. Equation (9) produces a differential pressure measurement, which may be useful for replacing an absolute pressure sensor in other systems.

In some examples, the flow is not laminar due to flow element dimensions, high pressures, or the flow element not being long enough to have a fully developed flow profile. In those examples, the Darcy-Weisbach equation may be more precise, because a friction factor is included in the equation to consider the flow regime and produce a closer approximation. In a cylindrical pipe of uniform diameter D, flowing full, the pressure loss due to viscous effects Δp is proportional to length L and can be characterized by the Darcy-Weisbach equation (Equation (10)):

$$\frac{Δ ρ}{L} = {f}_{D} \cdot \frac{ρ}{2} \cdot \frac{{〈 v 〉}^{2}}{{D}_{H}} \qquad ( 10 )$$

where the pressure loss per unit length

$$( \frac{Δ ρ}{L} )$$

in Pascals per meter is a function of:
ρ, the density of the fluid (kg/m³);
D_{H}, the hydraulic diameter of the pipe in meters (for a pipe of circular section, this equals D; otherwise D_{H}=4A/P for a pipe of cross-sectional area A and perimeter P);
(v), the mean flow velocity, experimentally measured as the volumetric flow rate Q per unit cross-sectional wetted area (m/s); and
f_{D}, the Darcy friction factor, which is a dimensionless quantity that describes the friction losses in pipe flow.

Any model may be used for the pressure that approximates well the flow profile. Equation (9) may work well at low flow rates for applications such as oxygen concentrators. For ventilators and CPAP machines, Equation (10) may provide more accurate results.

The examples herein provide quick response times, as ultrasonic measurements are generally faster than other measurements, such as chemical measurements. Ultrasonic transducers are also longer lasting than many other types of sensors. A properly designed flow element 110 produces a laminar flow, which is a consistent flow through the cross section of flow element 110 so that flow may be measured accurately. A laminar flow means the flow along the edges of flow element 110 is the same or similar to the flow through the center of the flow element 110.

As described above, pressure, concentration, and flow may be measured with ultrasonic transducers 120. Therefore, fewer sensors are used in system 100 compared to existing systems, which reduces size and cost. In examples herein, CO₂ is also measured. The speed of sound C is related to concentration in a known binary gas mixture. Therefore, if the percentage of O₂ in a gas mixture is known (such as when air and O₂ are provided to a patient via cannula 104), the percentage of CO₂ may be determined by measuring the speed of sound in the gas mixture using ultrasonic transducers 120.

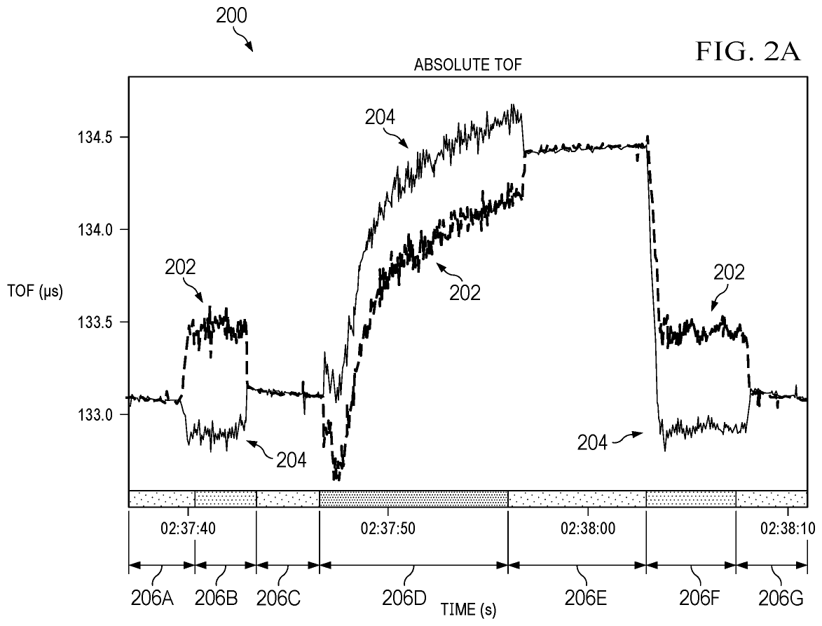

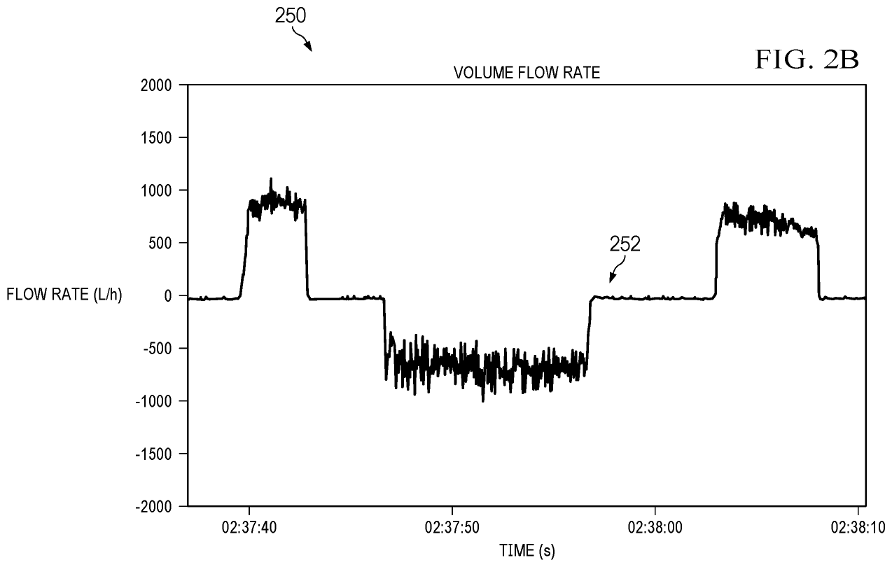

FIGS. 2A and 2B are example graphs for measuring CO₂ concentration. Graph 200 in FIG. 2A illustrates example time of flight measurements for ultrasonic transducers 120 while a patient is inhaling and exhaling a known O₂ mixture. Graph 250 in FIG. 2B illustrates example volume flow rates during inhalation and exhalation.

In FIG. 2A, the x-axis shows time in seconds. The y-axis shows the absolute TOF in microseconds (μs), as measured by ultrasonic transducers 120. FIG. 2A shows the TOF for a patient inhaling and exhaling a known O₂ mixture. In FIG. 2A, curve 202 is the TOF for a first ultrasonic transducer (such as 120A), and curve 204 is the TOF for a second ultrasonic transducer (such as 120B). Various time intervals 206A to 206G are shown on the x-axis.

During time interval 206A, the patient is neither inhaling or exhaling. During this interval, the air flow in flow element 110 is minimal, so the TOF measured by ultrasonic transducers 120A and 120B is roughly the same. Therefore, curves 202 and 204 coincide during interval 206A, with a TOF of about 133.1 μs.

During interval 206B, the patient is inhaling. During inhalation, the ultrasonic transducers 120A and 120B produce different TOF values. One ultrasonic transducer 120 is measuring TOF with the flow of air, and the other is measuring TOF against the flow of air. Therefore, the ultrasonic transducers produce different TOFs during interval 206B. In interval 206B, the ultrasonic transducer 120 that is measuring against the flow of air produces curve 202. The ultrasonic transducer 120 that is measuring with the flow of air produces curve 204. During interval 206B, curve 204 is lower than curve 202, which means curve 204 measures a lower TOF (e.g., a faster measurement that is going with the flow of air). During interval 206B, curve 202 is higher than curve 204, which means curve 202 measures a higher TOF (e.g., a slower measurement that is going against the flow of air).

During interval 206C, the patient is neither inhaling or exhaling, so the TOF measured by ultrasonic transducers 120A and 120B is roughly the same. Therefore, curves 202 and 204 coincide during interval 206C, with a TOF of about 133.1 μs.

During interval 206D, the patient is exhaling. During exhalation, the air flow is in the opposite direction than during inhalation. Therefore, during interval 206D, curve 204 is above curve 202. Curve 204 measures a higher TOF because the ultrasonic transducer 120 associated with curve 204 is now measuring against the flow of air. Curve 202 measures a lower TOF because the ultrasonic transducer 120 associated with curve 202 is now measuring with the flow of air.

During interval 206E, the patient has finished exhaling, and the air flow is relatively idle. The TOF measured by ultrasonic transducers 120A and 120B is the same, and curves 202 and 204 coincide during interval 206E. As shown in FIG. 2A, the TOF during interval 206E is about 134.5 μs. This TOF is substantially higher than the TOF during interval 206C, where the air flow was also idle. The post-exhalation TOF during interval 206E is higher than interval 206C because the exhalation includes a larger concentration of CO₂ than the inhalation. In an example, a patient is inhaling an air mixture of 21% O₂ and air during interval 206C. The patient may exhale about 17% 02, 4% CO₂, and air during interval 206E. In a closed system, the difference in the TOF between intervals 206C and 206E may be measured to determine the amount of CO₂ in the exhalation. An example process for determining CO₂ concentration is described below.

FIG. 2A shows another inhalation at interval 206F. During interval 206F, the ultrasonic transducer 120 that is measuring against the flow of air produces curve 202. The ultrasonic transducer 120 that is measuring with the flow of air produces curve 204 (similar to interval 206B). Finally, at interval 206G, the air flow is idle and the TOFs measured by ultrasonic transducers 120A and 120B are similar.

In FIG. 2B the x-axis shows time in seconds and the y-axis shows the volume flow rate in liters per hour. The time shown on the x-axis in FIG. 2B corresponds to the time shown on the x-axis in FIG. 2A. Therefore, curve 252 shows the volume flow rate during the inhalation and exhalation periods described above with respect to FIG. 2.

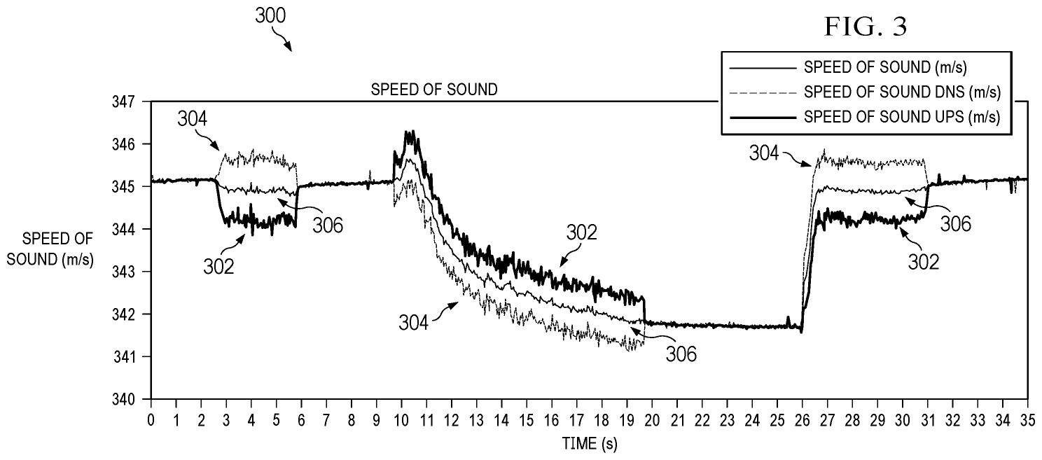

FIG. 3 is a graph 300 illustrating an example speed of sound during inhalation and exhalation. In graph 300, the x-axis represents time in seconds, and the y-axis represents the speed of sound in m/s. The curve 302 represents the speed of sound measurement for a first ultrasonic transducer 120 (e.g., upstream). The curve 304 represents the speed of sound measurement for a second ultrasonic transducer 120 (e.g., downstream). The curve 306 represents the speed of sound measurement for the medium itself, agnostic of the flow. Curve 306 represents the derived speed of sound of the medium, which removes the speed of sound additions due to the flow. During inhalation (between times 3 to 6 seconds, and between times 26 to 31 seconds), curve 304 measures a higher speed of sound than curve 302, so the second ultrasonic transducer 120 is measuring with the flow of air during these time intervals. During exhalation (between times 10 and 20 seconds), curve 302 measures a higher speed of sound than curve 304, so the first ultrasonic transducer 120 is measuring with the flow of air during these time intervals. Curve 306 shows that the speed of sound for the medium lies between curves 302 and 304.

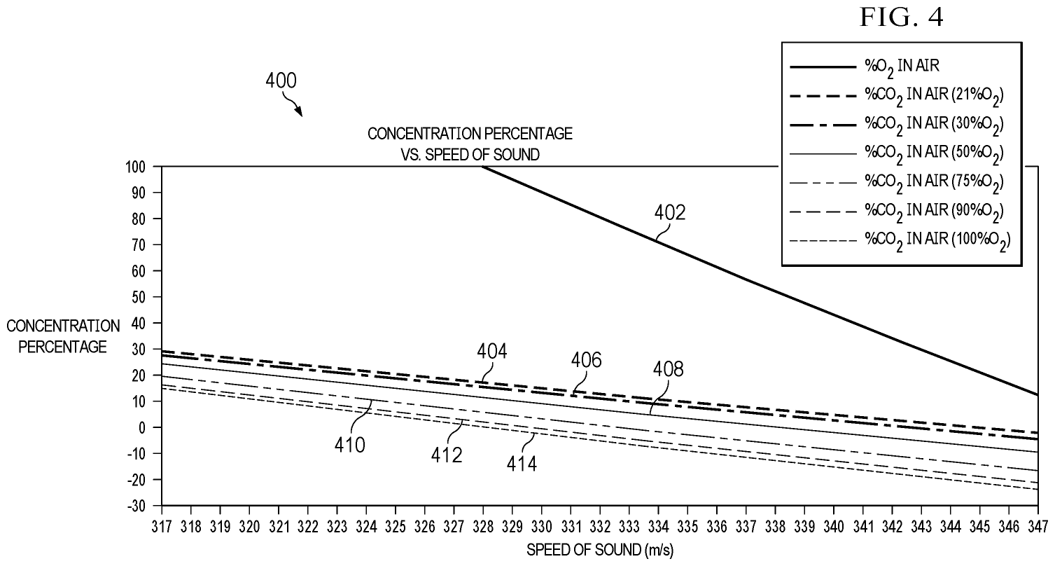

In this example, the average speed during inhalation is approximately 344.8 m/s. The average speed during exhalation is approximately 341.7 m/s. With these measurements, and the graph shown in FIG. 4 described below, the concentration of CO₂ may be determined.

FIG. 4 is a graph 400 illustrating an example concentration percentage versus speed of sound. As described above, if the O₂ concentration is known in an air mixture, the speed of sound may be measured to determine the CO₂ concentration. In graph 400, the x-axis represents the speed of sound, and the y-axis represents the concentration percentage. The curves on graph 400 show the different concentrations of O₂ or CO₂ in air, where air is the normal mixture of N₂ and O₂ in air.

Graph 400 includes curves 402, 404, 406, 408, 410, 412, and 414. Curve 402 represents the O₂ percentage in an air mixture. As an example, at about 80% O₂ concentration, the speed of sound is approximately 332 m/s. In an example, the formula for curve 402 is y=−4.82x+1684.

Curves 404, 406, 408, 410, 412, and 414 represent the CO₂ percentage in different air mixtures, where the different air mixtures include different O₂ concentrations. For example, curve 404 represents the percentage of CO₂ in air with 21% O₂. The formula for curve 404 is y=−1.054x+363.2. Curve 406 represents the percentage of CO₂ in air with 30% O₂. The formula for curve 406 is y=−1.077x+369.1. Curve 408 represents the percentage of CO₂ in air with 50% O₂. The formula for curve 408 is y=−1.133x+383.5. Curve 410 represents the percentage of CO₂ in air with 75% 02. The formula for curve 410 is y=−1.212x+403.8. Curve 412 represents the percentage of CO₂ in air with 90% O₂. The formula for curve 412 is y=−1.266x+417.5. Curve 414 represents the percentage of CO₂ in air with 100% O₂. The formula for curve 414 is y=−1.292x+424.4. As seen by these formulas, the curves have slightly different slopes m (where the slope m is found in y=mx+b).

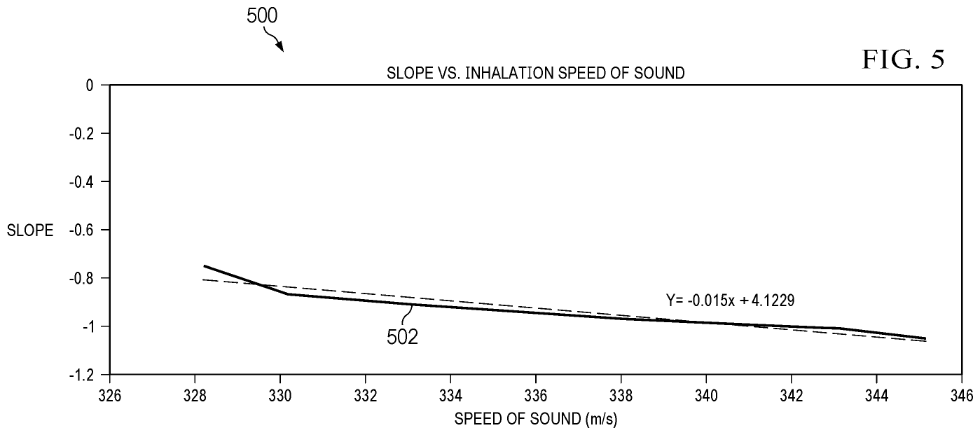

The curves 404 to 414 in FIG. 4 may be useful for determining the CO₂ concentration. An example procedure is described with respect to FIG. 5 below.

FIG. 5 is a graph 500 illustrating example slope versus inhalation speed of sound. Graph 500 shows the relationship for the slope of the curves 404 to 414 in FIG. 4. With this slope, the CO₂ concentration may be measured. In graph 500, the x-axis represents the speed of sound in m/s, and the y-axis represents the slope. The curve 502 represents the slope versus the inhalation speed of sound, and can be approximated by the formula y=−0.015x+4.1229.

In one example, the change in CO₂ concentration (Δy) is found by multiplying the slope m (shown in FIG. 5, and based on the O₂ concentration) by the change in the speed of sound between exhalation and inhalation (Δx), measured in m/s. Therefore, Δy=mΔx. If the inhalation speed is 344.82 m/s, and the exhalation speed is 341.72 m/s, the Δx is −3.10 m/s (exhalation minus inhalation). The slope m depends on the inhalation speed of sound, which is 344.82 m/s. Graph 500 provides the slope m, which is the y-axis of graph 500. Based on graph 500 and the curve 502 with a formula y=−0.015x+4.1229, where x is 344.82 m/s, y equals −1.0494 (e.g., the y-axis term), which is the slope m. Then, Δy=mΔx. Here, Δx is −3.10, and m is −1.0494. Therefore, Δy is 3.253. The percentage change in CO₂ concentration is 3.253% in between exhalation and inhalation in this example. In examples described herein, CO₂ concentration is measured in an air mixture if the O₂ concentration is known. Ultrasonic transducers 120 can also replace a CO₂ sensor in examples described herein.

In an example, the method for determining exhaled CO₂ from the difference between inhaled versus exhaled speed of sound is also used in the cases where the base gas mixture is binary or unknown. The binary method may be used for inhaled CO₂ if the mixture is known and binary. The delta between the inhaled and exhaled speed of sound plus the slope curves described herein may be used to determine exhaled CO₂, even if the base gas is not known. This process may provide a good metric of respiratory exchange ratio.

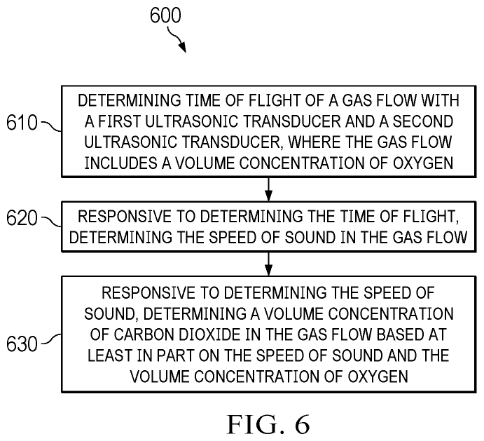

FIG. 6 is a flow diagram of an example method 600 for determining volume concentration. The steps of method 600 may be performed in any suitable order. The hardware components described above with respect to FIGS. 1A and 1B performs method 600 in some examples. Any suitable hardware, software, or digital logic performs method 600 in some examples.

Method 600 begins at 610, where a processor or controller determines time of flight of a gas flow with a first ultrasonic transducer and a second ultrasonic transducer, where the gas flow includes a volume concentration of oxygen. As described above, TOF measurements may be collected by ultrasonic transducers 120. The differential TOF measurements may then be used for determining other qualities of the gas flow as described with the equations above.

Method 600 continues at 620, where, responsive to determining the time of flight, determining a speed of sound in the gas flow. As described above, the velocity of the gas flow may be determined, and then the speed of sound C may be determined from the velocity.

Method 600 continues at 630, where, responsive to determining the speed of sound, determining a volume concentration of carbon dioxide in the gas flow based at least in part on the speed of sound and the volume concentration of oxygen. As described above, if the volume concentration of O₂ is known, the graphs and equations described above may be useful for determining the volume concentration of CO₂. Therefore, with a temperature sensor 122 and ultrasonic transducers 120, flow rate, O₂ concentration, and CO₂ concentration may be calculated. Differential pressure may also be calculated as described above, without the use of any additional sensors.

In examples herein, ultrasonic transducers 120 are used for flow sensing in the respiratory device. The ultrasonic transducers 120 perform TOF measurements for flow sensing. With a combination of absolute and differential TOF, temperature sensing, and humidity sensing, the systems and methods described herein determine O₂ concentration, CO₂ concentration, flow rate, and differential pressure. Therefore, the ultrasonic transducers 120, combined with a temperature and humidity sensor 122, can provide four different sense measurements. Separate sensors for each of these measurements may be replaced by the ultrasonic sensing system described herein. The systems and methods described herein may reduce size, reduce cost, increase efficiency, improve response time, and increase lifetime of a respiratory sensing system compared to previous solutions.

In this description, the term “couple” may cover connections, communications, or signal paths that enable a functional relationship consistent with this description. For example, if device A generates a signal to control device B to perform an action: (a) in a first example, device A is coupled to device B by direct connection; or (b) in a second example, device A is coupled to device B through intervening component C if intervening component C does not alter the functional relationship between device A and device B, such that device B is controlled by device A via the control signal generated by device A.

A device that is “configured to” perform a task or function may be configured (e.g., programmed and/or hardwired) at a time of manufacturing by a manufacturer to perform the function and/or may be configurable (or reconfigurable) by a user after manufacturing to perform the function and/or other additional or alternative functions. The configuring may be through firmware and/or software programming of the device, through a construction and/or layout of hardware components and interconnections of the device, or a combination thereof.

In this description, unless otherwise stated, “about,” “approximately” or “substantially” preceding a parameter means being within +/−10 percent of that parameter. Modifications are possible in the described examples, and other examples are possible within the scope of the claims.

What is claimed is:

1. A method, comprising:
determining time of flight of a gas flow with a first ultrasonic transducer and a second ultrasonic transducer, wherein the gas flow includes a volume concentration of oxygen;
responsive to determining the time of flight, determining a speed of sound in the gas flow; and
responsive to determining the speed of sound, determining a volume concentration of carbon dioxide in the gas flow based at least in part on the speed of sound and the volume concentration of oxygen.

2. The method of claim 1, further comprising:
determining the volume concentration of carbon dioxide in the gas flow based at least in part on temperature.

3. The method of claim 1, further comprising:
determining the volume concentration of carbon dioxide in the gas flow based at least in part on humidity.

4. The method of claim 1, further comprising:
determining a flow rate of the gas flow with the first ultrasonic transducer and the second ultrasonic transducer; and
determining a pressure of the gas flow based at least in part on the flow rate.

5. The method of claim 4, wherein determining the pressure of the gas flow includes determining the pressure based at least in part on a length and a radius of a flow element that directs the gas flow.

6. The method of claim 1, wherein determining the speed of sound in the gas flow includes determining an inhalation speed of sound and an exhalation speed of sound.

7. A system, comprising:
a flow element configured to direct a gas flow;
a temperature sensor coupled to the flow element, the temperature sensor configured to determine a temperature of the gas flow;
a first ultrasonic transducer and a second ultrasonic transducer coupled to the flow element, wherein the first ultrasonic transducer and the second ultrasonic transducer are configured to determine a time of flight of the gas flow; and
a processor configured to determine a volume concentration of a gas in the gas flow based at least in part of the time of flight and the temperature of the gas flow.

8. The system of claim 7, wherein the gas is oxygen.

9. The system of claim 7, wherein the gas is carbon dioxide.

10. The system of claim 7, further comprising:
a humidity sensor configured to determine a humidity of the gas flow.

11. The system of claim 7, wherein the processor is further configured to determine a pressure of the gas flow based at least in part on the time of flight of the gas flow.

12. The system of claim 11, wherein the processor is further configured to determine the pressure of the gas flow based at least in part on a radius and a length of the flow element.

13. The system of claim 7, wherein the processor is further configured to determine a speed of sound in the gas flow based at least in part on the time of flight of the gas flow.

14. A system, comprising:
a respiratory device;
a flow element coupled to the respiratory device and configured to direct a gas flow, wherein the gas flow includes a volume concentration of oxygen;
a temperature sensor coupled to the flow element, the temperature sensor configured to determine a temperature of the gas flow;
a first ultrasonic transducer and a second ultrasonic transducer coupled to the flow element, wherein the first ultrasonic transducer and the second ultrasonic transducer are configured to determine time of flight of the gas flow;
a processor configured to determine a speed of sound in the gas flow; and
wherein, responsive to determining the speed of sound, the processor is further configured to determine a volume concentration of carbon dioxide in the gas flow based at least in part on the speed of sound and the volume concentration of oxygen.

15. The system of claim 14, wherein the respiratory device is a ventilator.

16. The system of claim 14, wherein the respiratory device is a continuous positive airway pressure (CPAP) machine.

17. The system of claim 14, wherein the processor is further configured to determine the volume concentration of carbon dioxide in the gas flow based at least in part on temperature.

18. The system of claim 14, wherein the processor is further configured to:
determine a flow rate of the gas flow with the first ultrasonic transducer and the second ultrasonic transducer; and
determine a pressure of the gas flow based at least in part on the flow rate.

19. The system of claim 18, wherein determining the pressure of the gas flow includes determining the pressure based at least in part on a length and a radius of the flow element.

20. The system of claim 14, wherein the processor is further configured to determine the speed of sound in the gas flow based at least in part on the time of flight of the gas flow.
